(19) United States
(12) Patent Application Publication
(10) Pub. No.: US 2006/0154953 A1
(43) Pub. Date: Jul. 13, 2006

(54) Amorphous tacrolimus and preparation thereof

(76) Inventors: Vilmos Keri, Debrecen (HU); Adrienne Kovacsne-Mezei, Debrecen (HU); Andrea Csorvasi, Debrecen (HU); Erzsebet Meszaros Sos, Debrecen (HU)

Correspondence Address: KENYON & KENYON LLP, ONE BROADWAY, NEW YORK, NY, 10004, US

(21) Appl. No.: 11/326,724

(22) Filed: Jan. 5, 2006

Related U.S. Application Data
(60) Provisional application No. 60/641,868, filed on Jan. 5, 2005.
(60) Provisional application No. 60/705,681, filed on Aug. 3, 2005.

(51) Int. Cl. A61K 31/4745

(52) U.S. Cl. 514/291; 540/456

(57) ABSTRACT

The present invention provides amorphous tacrolimus in a free drug particulate form. Also provided are methods for preparing amorphous tacrolimus, and a tablet containing amorphous tacrolimus.

### RELATED APPLICATIONS

This application claims the benefits of U.S. Provisional Applications Ser. Nos. 60/641,868 and 60/705,681, filed Jan. 5, 2005, and Aug. 3, 2005, respectively, the contents of which are incorporated herein by reference in their entirety.

### FIELD OF THE INVENTION

The present invention is directed to amorphous tacrolimus in a free drug particulate form, novel methods of producing amorphous tacrolimus, and a tablet comprising amorphous tacrolimus.

### BACKGROUND OF THE INVENTION

Macrolides are multi-membered lactone rings having one or more deoxy sugars as substituents. Erythromycin, azithromycin, and clarithromycin are macrolides that have bacteriostatic and/or bactericidal activity. Ascomycin, tacrolimus, and Pimecrolimus are also macrolides.

Tacrolimus (FK 506) is a macrolide antibiotic that is also an immunosuppressive agent, produced by Streptomyces tsukubaensis. Its chemical name is 3S-[3R*[E(1S*,3S *,4S*)],4S*,5R*,8S*,9E,12R*,14R*,15S*,16R*,18S*,19S*,26aR*]]-5,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido [2,1-c][1,4]oxaazacyclotricosine-1,7,20,21(4H,23H)-tetrone, monohydrate, and it has the chemical formula:

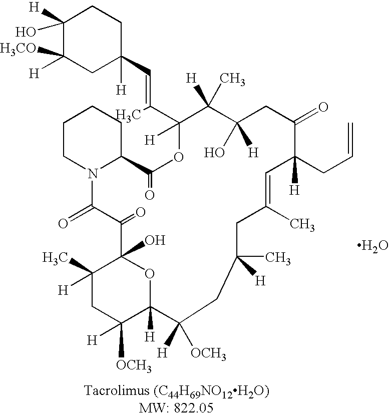

More potent than cyclosporin, tacrolimus has a selective inhibitory effect on T-lymphocytes.

The invention relates to the solid state physical properties of tacrolimus. These properties may be influenced by controlling the conditions under which tacrolimus is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants, such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered active ingredient may reach the patient's bloodstream. The rate of dissolution is also a consideration in formulating syrups, elixirs and other liquid medicaments. The solid state form of a compound may also affect its behavior on compaction and its storage stability.

The discovery of new forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for additional forms of tacrolimus and/or processes for their preparation.

### SUMMARY OF THE INVENTION

In one embodiment, the present invention provides amorphous tacrolimus in a in a free drug particulate form. This amorphous tacrolimus preferably contains not more than about 5% of the crystalline form of tacrolimus characterized by a powder XRD having peaks at about 10.5, 11.3 and 13.8±0.2 degrees 2θ.

In another embodiment, the present invention provides a method of preparing amorphous tacrolimus, comprising dissolving tacrolimus in an organic polar solvent, and removing the organic polar solvent. This process may be repeated, resulting in amorphous tacrolimus.

In yet another embodiment, the present invention provides a pharmaceutical formulation comprising amorphous tacrolimus in a free drug particulate form, and a pharmaceutically acceptable excipient.

In one embodiment, the present invention provides a tablet comprising amorphous tacrolimus.

In a further embodiment, the present invention provides a tablet comprising amorphous tacrolimus containing no more than about 5% of the crystalline form of tacrolimus, characterized by a powder XRD having peaks at about 10.5, 11.3 and 13.8±0.2 degrees 2θ.

In yet another embodiment, the present invention provides a method for treating a patient suffering from gram positive bacterial infection, comprising the step of administering to the patient the above pharmaceutical formulation. Also provided is a method of providing immunosuppression to a patient in need thereof comprising the step of administering to the patient the above pharmaceutical formulation.

### BRIEF DECRIPTION OF THE DRAWINGS

FIG. 1 illustrates a powder X-Ray Diffraction pattern of amorphous tacrolimus in a free drug particulate form;

FIG. 2 illustrates a powder X-Ray Diffraction pattern of amorphous tacrolimus in a free drug particulate form, obtained in Example 2; and

FIG. 3 illustrates a microscope image of amorphous tacrolimus in a free drug particulate form.

### DETAILED DESCRIPTION

As used herein, the term “room temperature” refers to a temperature of about 15° C. to about 30° C., preferably about 18° C. to about 25° C.

As used herein, the term “free drug” refers to solid particles not intimately embedded in a coprecipitate.

As used herein, the term “particulate” refers to one or more individual particles.

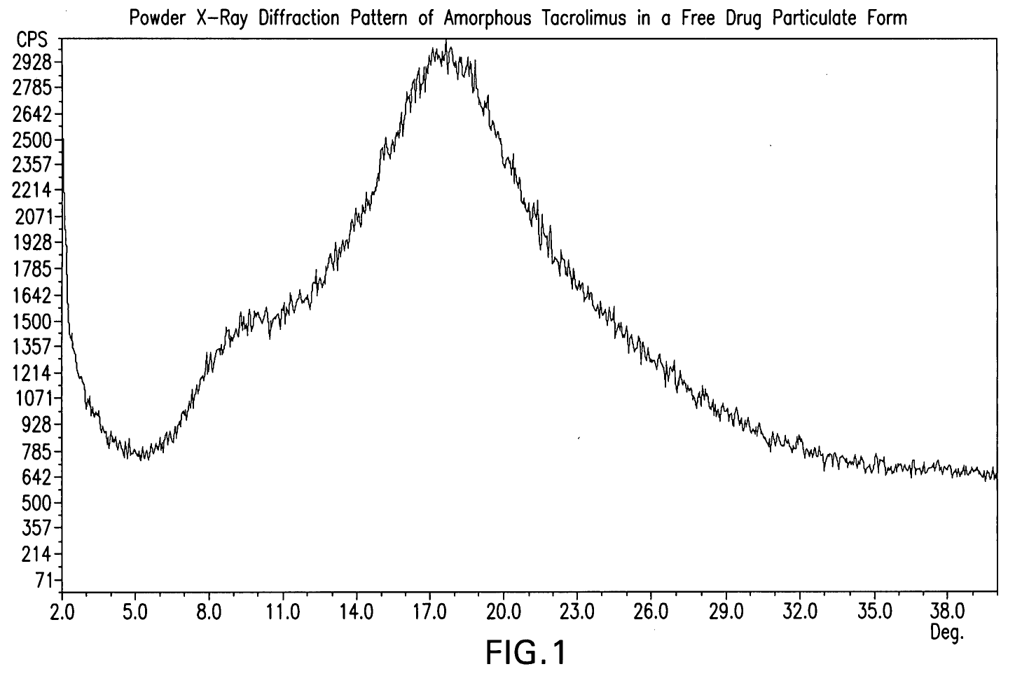

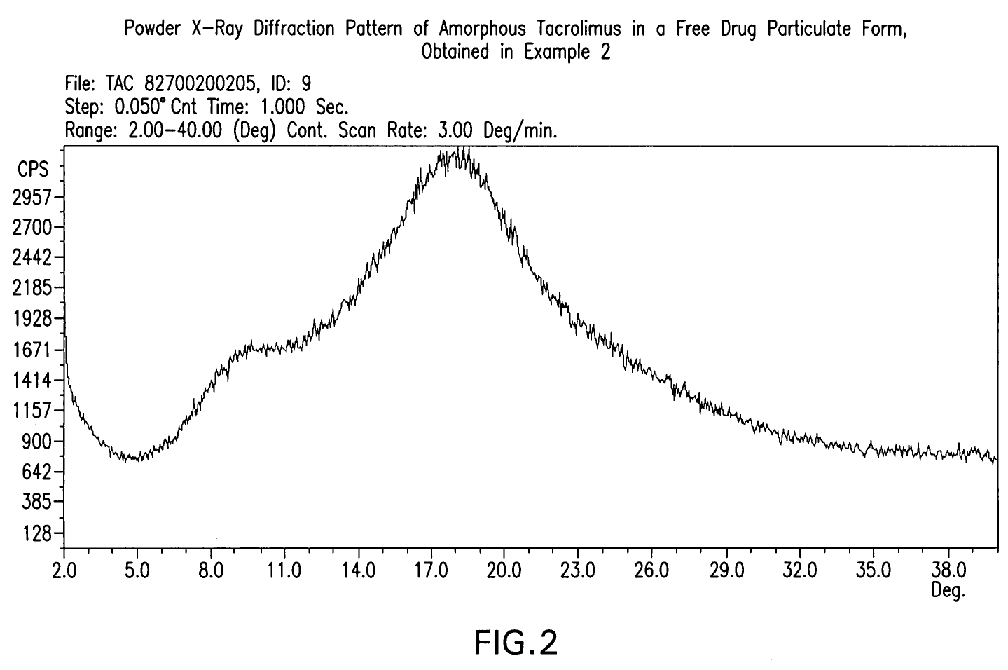

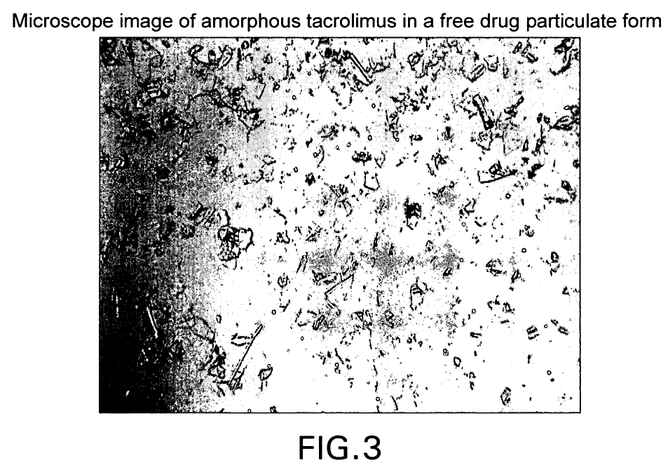

The present invention provides amorphous tacrolimus in a free drug particulate form. The amorphous tacrolimus of the present invention is characterized by the powder X-Ray Diffraction pattern, substantially as depicted in FIGS. 1 and 2. A microscope image of amorphous tacrolimus in a free drug particulate form is shown in FIG. 3.

The amorphous tacrolimus of the present invention contains no more than about 5% of the crystalline form of tacrolimus, characterized by a powder XRD having peaks at about 10.5, 11.3 and 13.8±0.2 degrees 2θ. Preferably, the amorphous tacrolimus of the present invention contains no more than about 3% of the crystalline form described above, and, most preferably, the amorphous tacrolimus of the present invention contains no more than about 1% of that crystalline form. Amorphous tacrolimus has better physical properties than the crystalline tacrolimus, e.g., improved dissolution and/or solubility.

The presence of crystalline tacrolimus in the amorphous form, as a bulk active ingredient, or in the pharmaceutical compositions may be observed using known methods, such as X-ray powder diffraction or solid-state ¹³C-NMR. Any instrumentation of X-Ray powder diffraction or solid-state NMR normally available in laboratories is suitable for monitoring crystalline tacrolimus in amorphous tacrolimus as a bulk or in pharmaceutical compositions.

Use of tacrolimus in a free drug particulate form is advantageous over a coprecipitate, in that the particle size distribution can be controlled.

The present invention further provides a novel method of producing amorphous tacrolimus, comprising: dissolving tacrolimus in an organic polar solvent, and removing the organic polar solvent to obtain amorphous tacrolimus. This process may be repeated.

Preferably, the organic polar solvent is selected from the group consisting of C₁₋₆ alcohols, such as methanol, ethanol, n-propanol, iso-propanol, n-butanol, iso-butanol, tert-butanol and 2-butanol; C₃₋₈ alkyl esters, such as ethyl acetate, isobutyl acetate, n-butyl acetate, ethylformate, n-propyl acetate and iso-propyl acetate; C₂₋₈ alkyl ketones, such as acetone and methyl ethyl ketone, C₂₋₈ alkyl ethers, such as tetrahydrofuran; acetonitrile; and mixtures thereof. Most preferably, the organic polar solvent is ethyl acetate or acetone.

Preferably, the organic polar solvent may be removed by any method known in the art, such as evaporation. Preferably, the organic polar solvent is evaporated, followed by further cooling to about room temperature.

The evaporation is preferably done to dryness. The solution may be evaporated in a rotavapor under reduced pressure, or under atmospheric pressure. The evaporation may be performed in any heated vessel under reduced pressure, such as a test tube of a rotavapor, a commercial test tube, or any other glass reactor under atmospheric or reduced pressure.

The solution may be placed in the vessel all at once, by gradual addition or continuously into a heated equipment under reduced pressure. Preferably, the heating capacity and the addition rate are equal, or the heating capacity is higher than the addition rate, in order to get the dried foam of amorphous tacrolimus. When the heating capacity is insufficient to remove the solvent at once, a further distillation of the solvent content may be required.

The continuous solution input can be ensured by injection using a pump or a capillary tube under reduced pressure. The capillary tube allows a slow intake of the solution into the vessel. Any tube of small diameter can be used instead of the capillary.

The evaporation process is preferably performed at a temperature of about 40° C. to about 60° C.

The present invention provides a pharmaceutical formulation comprising amorphous tacrolimus in a free drug particulate form and a pharmaceutically acceptable excipient.

The present invention also provides a tablet comprising amorphous tacrolimus.

The present invention further provides a tablet comprising amorphous tacrolimus containing no more than about 5% of the crystalline form of tacrolimus, characterized by a powder XRD having peaks at about 10.5, 11.3 and 13.8±0.2 degrees 2θ. Preferably, the tablet comprises amorphous tacrolimus containing no more than about 3% of the crystalline form of tacrolimus described above. Most preferably, the tablet comprises amorphous tacrolimus containing no more than about 1% of that crystalline form of tacrolimus.

The present invention also provides a method for treating a patient suffering from gram positive bacterial infection, comprising the step of administering to the patient the pharmaceutical formulation comprising a therapeutically effective amount of amorphous tacrolimus in a free drug particulate form. A further embodiment of the present invention is a method of providing immunosuppression to a patient in need thereof comprising the step of administering to the patient the pharmaceutical formulation comprising a therapeutically effective amount of amorphous tacrolimus in a free drug particulate form.

“Therapeutically effective amount” means the amount of the amorphous form that, when administered to a patient for treating a disease or other undesirable medical condition, is sufficient to have a beneficial effect with respect to that disease or condition. The “therapeutically effective amount” will vary depending on the disease or condition and its severity, and the age, weight, etc., of the patient to be treated. Determining the therapeutically effective amount of a given amorphous form is within the ordinary skill of the art and requires no more than routine experimentation.

In addition to the active ingredient(s), the pharmaceutical formulations of the present invention may contain one or more excipients. Excipients are added to the formulation for a variety of purposes.

Diluents may be added to the formulations of the present invention. Diluents increase the bulk of a solid pharmaceutical composition, and may make a pharmaceutical dosage form containing the composition easier for the patient and care giver to handle. Diluents for solid compositions include, for example, microcrystalline cellulose (e.g., AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatin, guar gum, hydrogenated vegetable oil, hydroxyethyl cellulose, hydroxypropyl cellulose (e.g., KLUCEL®), hydroxypropyl methyl cellulose (e.g., METHOCEL®), liquid glucose, magnesium aluminum silicate, maltodextrin, methylcellulose, polymethacrylates, povidone (e.g., KOLLIDON®, PLASDONE®), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g., AC-DI-SOL®, PRIMELLOSE®), colloidal silicon dioxide, croscarmellose sodium, crospovidone (e.g., KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB®), and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form, such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

The present invention is not intended to encompass true solutions of tacrolimus whereupon the structure of the novel form and the properties that distinguish the novel form of tacrolimus of the present invention are lost. Thus, the pharmaceutical compositions of the present invention comprising the novel amorphous form of tacrolimus disclosed herein will primarily be solid pharmaceutical compositions. However, the use of the novel forms to prepare solutions (e.g., so as to deliver tacrolimus in a liquid pharmaceutical formulation) is considered to be within the contemplation of the invention.

In liquid pharmaceutical compositions prepared using the amorphous form of the present invention, tacrolimus and any other solid excipients are dissolved or suspended in a liquid carrier, such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents, such as sorbitol, saccharin, sodium saccharin, sucrose, aspartame, fructose, mannitol, and invert sugar may be added to improve the taste.

Preservatives and chelating agents, such as alcohol, sodium benzoate, butylated hydroxyl toluene, butylated hydroxyanisole, and ethylenediamine tetraacetic acid may be added at levels safe for ingestion to improve storage stability.

A liquid composition may also contain a buffer, such as gluconic acid, lactic acid, citric acid or acetic acid, sodium gluconate, sodium lactate, sodium citrate, or sodium acetate. Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The active ingredient and excipients may be formulated into compositions and dosage forms according to methods known in the art.

The amorphous form of the present invention may be used in pharmaceutical formulations or compositions as single components or mixtures together with other forms of tacrolimus. However, it is preferred that the pharmaceutical formulations or compositions of the present invention contain 25-100% by weight, especially 50-100% by weight, of the novel form, based on the total amount of tacrolimus in the formulation or composition. Preferably, such an amount of the novel form of tacrolimus is 75-100% by weight, especially 90-100% by weight. Highly preferred is an amount of 95-100% by weight.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

Instruments

The amorphous form of tacrolimus, produced by the methods of the present invention can be analyzed by Powder X-ray diffraction (PXRD) was performed on an X-Ray powder diffractometer, ARL, θ-θgoniometer, Cu-tube, solid state detector with Peltier cooling. The sample holder was a round standard aluminum sample holder with round zero background. Scanning parameters: Range: 2-40 degrees 2θ, Continuous Scan, Rate: 3 degrees/min.

Amorphous tacrolimus can also be analyzed by thermal analysis, which can be carried out by digital scanning calorimetry (DSC) and by thermogravimetric analysis (TGA). DSC thermograms can be obtained on a DSC822^{e} Mettler Toledo instrument (Advanced Instruments, San Juan, Puerto Rico). Sample weight: 3-5mg; Heating rate: 10° C./min; Number of holes in the crucible: 3. TGA thermograms can be obtained on a Mettler TGA/SDTA 851 instrument (Advanced Instruments, San Juan, Puerto Rico) using a standard Allumina pan. Sample weight: 7-15mg; Heating rate: 10° C./min.

### Example 1

Crystallized tacrolimus (2 g) was dissolved in ethyl acetate (6 ml) and evaporated to dryness. This process was repeated twice. The evaporated foamy material was cooled to room temperature. At room temperature, glittering white amorphous particles (2.00 g) were obtained.

### Example 2

Crystallized tacrolimus of 140 g was dissolved in 112 ml acetone. The solution was injected into an empty equipment, which was heated to a temperature of 40-60° C. The equipment was maintained under reduced pressure. Foaming material was observed and then dried at 35-45° C. under reduced pressure, until the acetone content of the product was less than 0.5 %. White amorphous product was obtained.

What is claimed:

1. Amorphous tacrolimus in a free drug particulate form.

2. The amorphous tacrolimus of claim 1, substantially as depicted in FIGS. 1 and 2.

3. The amorphous tacrolimus of claim 1, substantially as depicted in FIG. 3.

4. The amorphous tacrolimus of claim 1, containing no more than about 5% of a crystalline form of tacrolimus, wherein the crystalline form is characterized by a powder XRD pattern having peaks at about 10.5, 11.3 and 13.8±0.2 degrees 2θ.

5. The amorphous tacrolimus of claim 1, containing no more than about 3% of a crystalline form of tacrolimus, wherein the crystalline form is characterized by a powder XRD pattern having peaks at about 10.5, 11.3 and 13.8±0.2 degrees 2θ.

6. The amorphous tacrolimus of claim 1, containing no more than about 1% of a crystalline form of tacrolimus, wherein the crystalline form is characterized by a powder XRD pattern having peaks at about 10.5, 11.3 and 13.8±0.2 degrees 2θ.

7. A process for preparing amorphous tacrolimus, comprising dissolving tacrolimus in an organic polar solvent, and removing the organic polar solvent to obtain amorphous tacrolimus.

8. The process of claim 7, wherein the organic polar solvent is selected from the group consisting of C₁₋₆ alcohols, C₃₋₈ alkyl esters, C₂₋₈ alkyl ketones; C₂₋₈ alkyl ethers, acetonitrile, and mixtures thereof.

9. The process of claim 8, wherein the organic polar solvent is selected from the group consisting of methanol, ethanol, n-propanol, iso-propanol, n-butanol, iso-butanol, tert-butanol, 2-butanol, ethyl acetate, isobutyl acetate, n-butyl acetate, ethylformate, n-propyl acetate, iso-propyl acetate, acetone, methyl ethyl ketone, tetrahydrofuran, acetonitrile, and mixtures thereof.

10. The process of claim 9, wherein said organic polar solvent is ethyl acetate or acetone.

11. The process of claim 7, wherein the process is repeated.

12. The process of claim 7, wherein the solvent is removed by evaporation.

13. The process of claim 8, wherein the evaporation is performed in a rotavapor, a test tube of a rotavapor, or a commercial test tube.

14. The process of claim 8, wherein the evaporation is performed under atmospheric or reduced pressure.

15. The process of claim 14, wherein the evaporation is performed under reduced pressure.

16. The process of claim 8, wherein the evaporation is performed at a temperature of about 40° C. to about 60° C.

17. A pharmaceutical formulation comprising the amorphous tacrolimus of claim 1 and a pharmaceutically acceptable excipient.

18. A tablet comprising amorphous tacrolimus.

19. The tablet of claim 18, containing no more than about 5% of a crystalline form of tacrolimus, wherein the crystalline form is characterized by a powder XRD pattern having peaks at about 10.5, 11.3 and 13.8±0.2 degrees 2θ.

20. The tablet of claim 18, containing no more than about 3% of a crystalline form of tacrolimus, wherein the crystalline form is characterized by a powder XRD pattern having peaks at about 10.5, 11.3 and 13.8±0.2 degrees 2θ.

21. The tablet of claim 18, containing no more than about 1% of a crystalline form of tacrolimus, wherein the crystalline form is characterized by a powder XRD pattern having peaks at about 10.5, 11.3 and 13.8±0.2 degrees 2θ.

22. A method for treating a patient suffering from gram positive bacterial infection, comprising the step of administering to the patient the pharmaceutical formulation of claim 17.

23. A method for treating a patient suffering from gram positive bacterial infection, comprising the step of administering to the patient the tablet of claim 18.
